(19) United States
(12) Patent Application Publication
(10) Pub. No.: US 2023/0372140 A1
(43) Pub. Date: Nov. 23, 2023

(54) Universal ostomy bag with a irrigation system that prevents ballooning up ostomy bags and specializes in odor reducing ostomy bags

(71) Applicant: Myron Hester, Sacramento, CA (US)

(72) Inventor: Myron Hester, Sacramento, CA (US)

(21) Appl. No.: 17/746,993

(22) Filed: May 18, 2022

(51) Int. Cl. A61F 5/442; A61F 5/441

(52) U.S. Cl. CPC A61F 5/442; A61F 5/441; A61F 2005/4415

(57) ABSTRACT

The present invention discloses an advance ostomy bag which provides various novel advancements to the user. The assembly is easy to use and provides easy cleaning, fragrance addition, vent opening and closing system, gas chamber for sucking of gas and spill free structure.

### BACKGROUND

### Field of the Invention

The present invention relates to ostomy bags that can be comfortably worn by the patients and more particularly relates to a Universal ostomy bag with a irrigation system that prevents ballooning up ostomy bags and specializes in odor reducing ostomy bags with irrigation system.

### Description of the Related Art

An ostomy bag is a pouch that is employed to collect waste material that exits a person's body through a stoma, i.e. through an artificial, permanent, opening, surgically created in the ostomate's skin and connected to the intestine or to the bladder.

Colostomy, ileostomy, and urostomy bags, herein collectively referred to as ostomy bags, typically comprise a pouch of plastic, moisture-impermeable and odor-barrier, material; an opening in said bag to allow waste material to enter into the plastic envelope; and means to secure the bag in place with the opening connected to the stoma. This can be obtained by means of a tape that is heat bonded to the ostomy bag around the opening; said tape has an opening which is positioned in-line with the opening of the bag and the side of said tape which is adjacent to the ostomate's body is coated with an adhesive which allows adhesion of the ostomy appliance to the “peristomal area”, i.e., the skin area surrounding the stoma.

Preferably the means to secure the ostomy bag to the stoma and the ostomy bag itself, are constructed as two separable parts which are connected and disconnected by the user as desired. This is achieved for instance by applying to the ostomate's skin by means of a suitable adhesive a tape that bears on its outer surface a semi-rigid plastic snap ring, bonded by conventional means to the tape, that surrounds the opening. The ostomy bags then bear a second complementary snap ring mating with the tape one; said complementary snap ring is bonded to the ostomy bag and surrounds the opening therein. The user can therefore apply and remove the ostomy bag from the stoma without peeling off from the skin the means for securing the bag to the stoma. When assembled the snap ring provides a tight leak-free seal.

Today, such pouches are generally formed of light weight, odor proof, flexible polymeric materials and the collection systems are designed to be inconspicuous and permit the ostomate to engage in normal physical activity. However, many ostomates, particularly immediately following surgery, have fears concerning their ability to resume a “normal life”. These fears center around worries that the collection system will leak or that odor will escape and that the system will be noticeable even through their outer clothing. Part of these problems are due to the discharge of flatus into the pouch which can cause an embarrassing distention of the pouch.

In order to overcome the problem of gas build up within the pouch, it has been suggested that the pouch be provided with a tortuous path vent opening as note, for example, Baxter in U.S. Pat. No. 3,055,368 and Neumeier et al. in U.S. Pat. No. 4,185,630. Ornstein in U.S. Pat. No. 4,367,742 and Briggs et al. in U.K. Pat. application No. 2,031,282, for example, employ a tortuous path and a deodorizing filter. Other proposals have involved filters which could be attached to the pouch as note, for example, Steer et al. in U.S. Pat. No. 3,268,286 or filters attached over a vent opening in the pouch wall as note, for example, Bevan et al. U. K. Pat. application No. 2,083,760. It has also been suggested to employ a pouch having a deodorizing vent opening in its upper edge as note, for example, Steer et al. in U.S. Pat. No. 4,372,308, Kulbach et al. in U.S. Pat. No. 4,211,224, and Jensen in British Pat. No. 1,541,565. Bonfils in U.S. Pat. No. 3,439,677 disclose a pouch having a vent opening in the outer pouch wall opposite and above the stomal aperture covered by a deodorizing filter. Ockwell et al. in U.S. Pat. No. 4,120,715 and McLeod in U.S. Pat. No. 4,318,406 disclose multi-layered filters adapted to be sealed over a vent opening in the pouch wall. Steer et al. in U.S. Pat. No. 4,331,148 disclose employing a deodorizing cover bag.

Other venting pouch systems are disclosed by Nolan et al. in U.S. Pat. Nos. 3,759,260 and 3,804,091, by Nolan in U.S. Pat. No. 3,952,727, by Jessup et al. in U.S. Pat. No. 4,203,445, by Larsen et al. in British Pat. No. 1,571,382 and by LaGro in U.S. Pat. No. 4,274,848.

There are multiple solutions that have been presented in prior art in regarding urostomy bags. However, the utility of these systems has not been seen in advance form and for the above discussed needs. Looking at the prior art and market requirements, the need exists for an advancement that allows the user with comfort and ease of use. The proposed invention introduces a system which provides an advance irrigation system along the bag, which aims to provide various advancements to the user.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

It is a general object of the present invention to provide an improved form of ostomy pouch with irrigation system.

It is another object to provide an improved ostomy bag where the user is on the go without the fear of spill or mess.

Another object is to provide a configuration or construction that allows to release the gas out of bag without smell.

More specifically, it is the principal object of this invention is to provide a construction for an ostomy system such that the elderly and visually impaired may use the system with confidence.

It is also the objective of the system to provide an assembly which is designed to provide maximum benefit by providing irrigation key where it allows to inject water for easy cleaning.

According to another aspect, it is also the objective of the invention to provide an assembly which can be assembled or disassembled easily.

It is also the objective of the system to provide an assembly where the irrigation key can be used to add desired fragrance or nice smell.

It is further the objective of invention to provide simple connections of EZ flow valve key where once it is connected with irrigation key allows to keep vent opened or closed for gas release.

It is also the objective of the invention to provide an assembly which is of durable configuration made of high-quality material.

Other aspects, advantages and novel features of the present invention will become apparent from the detailed description of the invention when considered in conjunction with the accompanying drawings.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present invention is directed to provides a universal ostomy bag with multiple added functionalities.

The invention as per preferred embodiment provides an irrigation tube (B), Irrigation Straps (N), Irrigation Key (C), Universal Ostomy Bag (A), Ostomy drainage cap (E), EZ rest foldable bag (H), Gas chamber key (L), Gas chamber (F), Collapsible gas chamber (G), Foldable flush bag cap (O), Foldable flush bag (J), Flush bag (K), EZ flow valve key (D), EZ rest bag (H) and irrigation syringe (M).

The invention as per its illustrative embodiment presents an assembly where the Body side includes an aperture which can be enlarged so as to fit snugly around the stoma of the user. The stomal aperture is encircled by means for supporting the pouch on the body of the user. Conventionally, this support means is a faceplate which consists of a pressure sensitive medical grade adhesive material having a backing layer that is welded directly to the pouch wall.

As per its further embodiments, the aperture is located above stomal aperture in body side pouch wall. This reduces the possibility that fecal material discharged from the stoma will contact deodorizing insert. Pouch walls are selected from polymeric materials and laminates which possess the properties of being moisture impermeable, odor impermeable, and capable of being heat sealed or impulse welded. Suitable materials include polyethylene, a copolymer of vinyl chloride and polyvinylidene chloride, etc., and laminates thereof such as a laminate of ethylene vinylacetate or polyethylene and a copolymer of vinyl chloride and polyvinylidene chloride and a triple laminate composed of an outer and inner layer of ethylene vinylacetate and a core of a copolymer of vinyl chloride and polyvinylidene chloride. Pouch walls will be from about 2 to about 4 mils thick. The same materials are suitable for use as polymeric film cover. Polymeric film cover can be welded to outer pouch wall by heat welding or other techniques if the polymeric materials are compatible or the inner surface of cover can be coated with a pressure sensitive acrylic type adhesive.

The proposed assembly functionalities can be further understood by following details. By connecting the Irrigation key (C) with Gas chamber (L) will allow to release the gas out of ostomy bag. The Flush bag when connected to ostomy drainage cap will allows to clean ostomy bag.

As per further embodiments, by connecting Irrigation Syringe(M) to Irrigation Key will allow to inject water for easy cleaning. This will also allow to add favorite fragrances, By Connecting EZ rest foldable bah (H) to ostomy drainage cap will allow to sleep peacefully without the worry of gas buildups and leaks. The EZ flow valve key once connected to Irrigation key will allow to keep vent open and release the gas once desired.

The system as per its additional embodiments allows Gas chamber (F) to connect to Irrigation key to suck the gas from ostomy bag and release it at user's will.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1: An ostomy pouch comprising two pouch walls of moisture proof, odor proof polymeric materials sealed together along at least a substantial portion of their peripheral edge to form said pouch, one of said pouch walls having an aperture adapted in use to fit around the stoma of the user, said stomal aperture encircled by means for attaching said pouch to the body of the user, the second pouch wall having an aperture in its upper region above the stomal opening in the first pouch wall so that fecal matter discharged from the stoma into the pouch will not readily contact said second pouch wall aperture where the assembly comprises of:
Irrigation tube (B);
Irrigation Straps (N);
Irrigation Key (C);
Universal Ostomy Bag (A);
Ostomy drainage cap (E);
EZ rest foldable bag (H);
Gas chamber key (L);
Gas chamber (F);
Collapsible gas chamber (G);
Foldable flush bag cap (O);
Foldable flush bag (J);
Flush bag (K);
EZ flow valve key (D);
EZ rest bag (H); and,
Irrigation syringe (M).

2. The assembly as per claim 1, allows to release the gas out of ostomy bag without smell.

3. The assembly as per claim 1, allows easy cleaning and user can add favorite fragrance through Irrigation syringe.

4. The assembly as per claim 1, once connected to EZ rest bag user will be able to sleep conformably without the worry of gas buildup and leakage.

5. The assembly as per claim 1, allows the user to open or close the vents as desired.

6. The assembly as per claim 1, allows to such the gas out of ostomy bag and release it to location where the user desired.
